(19) United States
(12) Patent Application Publication
(10) Pub. No.: US 2010/0318039 A1
(43) Pub. Date: Dec. 16, 2010

(54) CONNECTORS

(75) Inventors: Kristian Hall, Hull (GB); Edward Hartwell, York (GB); Martin Richardson, Grimsby (GB)

Correspondence Address: FISH & RICHARDSON P.C., P.O. BOX 1022, MINNEAPOLIS, MN, 55440-1022, US

(73) Assignee: Smith & Nephew Plc, London (GB)

(21) Appl. No.: 12/808,451

(22) PCT Filed: Dec. 1, 2008
(86) PCT No.: PCT/GB2008/051136

§ 371 Date: Jun. 16, 2010

(30) Foreign Application Priority Data
Dec. 20, 2007 (GB) 0724827.1

(51) Int. Cl. A61M 39/22; A61M 39/10

(52) U.S. Cl. 604/246; 604/535

(57) ABSTRACT

A connector for joining ends of two conduits is described, the connector comprising: a male connector portion and a female connector portion, both portions having conduit connection means for the connection of a conduit to each portion; engagement means to hold the male and female connector portions in fluid flow establishing connection when connected together and valve means associated with said female portion.

The present invention relates to connectors for joining or separating two or more conduits particularly, though not exclusively, in the field of medical devices for establishing or stopping fluid flow through the conduits.

Many medical devices require the provision of conduits, particularly flexible conduits made from relatively soft plastics material tubing, for conveying various fluids to and from a patient. It is frequently necessary to interrupt the flow of fluid between a device and a patient in order to, for example, replace a source of fluid or to replace a receptacle receiving fluid from a patient, for example. An example of a device which requires fluid flow conduits to be provided between patient and device are topical negative pressure (TNP) therapy devices which are well known in the medical device art.

Frequently, conduits are merely pushed onto or pulled off a tubular spigot associated with the device to effect replacement of the device or item in question. In other instances conduits may be cut and clamps of various types used to seal off the open ends of the severed conduit.

It is a fact that patients, especially in hospitals for example, may have many conduits attached to them and it is important that connectors in conduits used to link medical devices to patients should be as foolproof as possible and that the connectors should have unique features so that devices cannot be misconnected to a patient. Similarly, a device may have a plurality of conduits conveying different fluids to and from a patient and it is important that such conduits cannot be crossed over or be wrongly connected. Furthermore, the ability to ensure correct connection of various fluids to required sites between patient and devices also helps to minimise the spread of biohazard and cross contamination.

It is desirable that a connector be provided in a conduit intermediate the device and patient so that the conduit may be broken, ideally without leakage of fluid from at least the device.

According to the present invention there is provided a connector for joining ends of two conduits, the connector comprising: a male connector portion and a female connector portion, both portions having conduit connection means for the connection of a conduit to each portion; engagement means to hold the male and female connector portions in fluid flow establishing connection when connected together and valve means associated with said female portion.

As stated above the connector according to the present invention is to connect two conduits together. The two conduits may be two conduit portions with one conduit portion attached to a wound dressing on a patient, for example, and the second conduit portion connected to a TNP therapy device, for example, which it desired to operably connect to the wound dressing.

The engagement means for holding the male and female portion in flow establishing engagement may comprise mutually engageable threaded portions on each part, for example, or resilient portions on the male and/or female connector portions which effect a snap-fit when the two portions are pushed together. In the latter case the two connector portions may be pulled apart by only a moderate force to effect disconnection.

The female connector portion may also have valve means associated there with so that when the male and female portions are engaged the valve means are either opened automatically by the act of engaging the male and female portions or may be opened manually after engagement.

Suitable valve means may include ball valves having a sealing ball which is resiliently biased into sealing engagement with a seat when the two connector portions are disengaged. Engagement causes the sealing ball to be moved out of engagement with its seat by, for example, a projection on the male portion which moves the ball on engagement of the male and female connector portions to establish a fluid flow channel through the engaged male and female connector portions.

Alternative valve means may comprise a resilient plastics material diaphragm valve portion which is opened on insertion of the male connector portion into the female connector portion. Such a resilient plastics material diaphragm portion may comprise a separate item in the construction of a connector according to the present invention or may be included as a part of one of a male or a female connector portion by means of a so-called 2-shot plastics injection moulding process, for example. Such a valve portion has the advantage that it may form an in-situ sealing gasket at the junction of the male and female connector portions and no further sealing means are required.

Further alternative valve means may include a manually operated valve which may be opened after the male and female connector portions have been connected. Such a valve may be opened or closed at will regardless of the state of connection of the male and female connector portions.

The male and female connector portions may be provided with suitable connection means such as hollow spigot portions for the connection of conduit portions to each connector portions. As is well known in the art such hollow spigot portions may be provided with suitable serrations such as fir-tree formations to aid retention of typical plastics conduit materials used in the art.

Advantageously, at least the bodies of the male and female connector portions may be produced by moulding of plastics material.

In order that the present invention may be more fully understood examples will now be described by way of illustration only with reference to the accompanying drawings, of which:

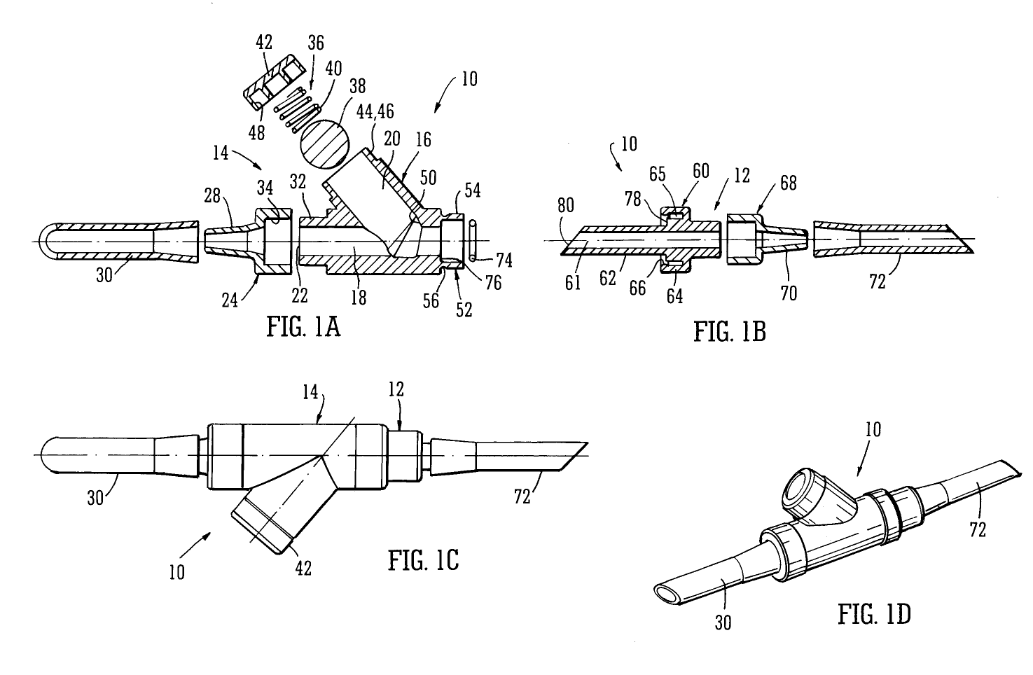

FIGS. 1A to 1D show various views of a first embodiment of a connector according to the present invention;

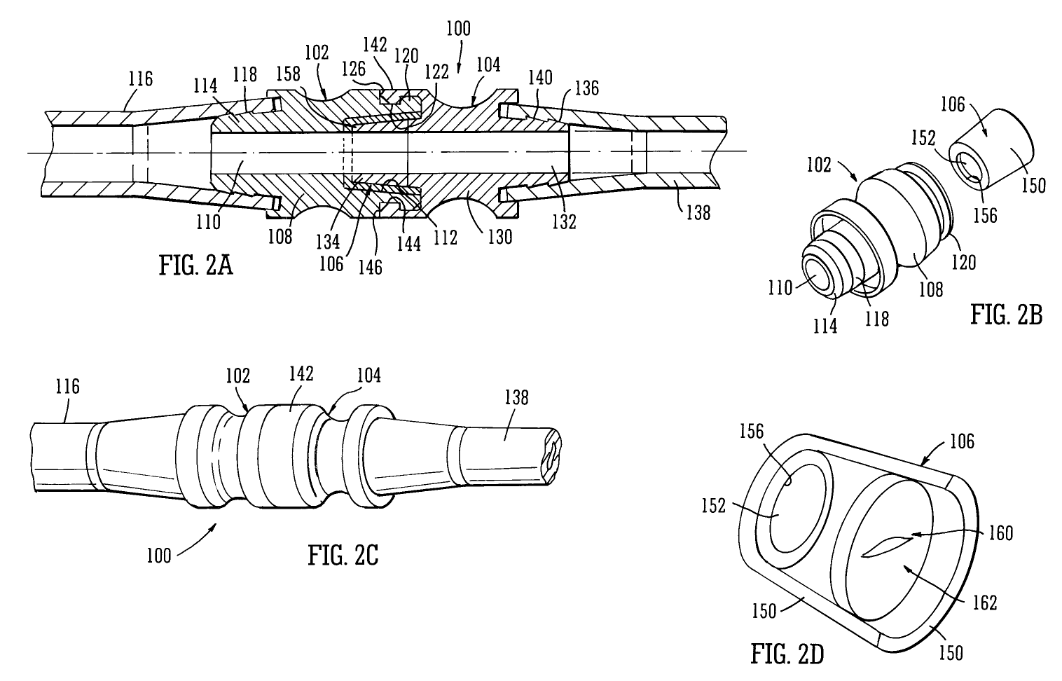

FIGS. 2 A to 2D show various views of a second embodiment of a connector according to the present invention;

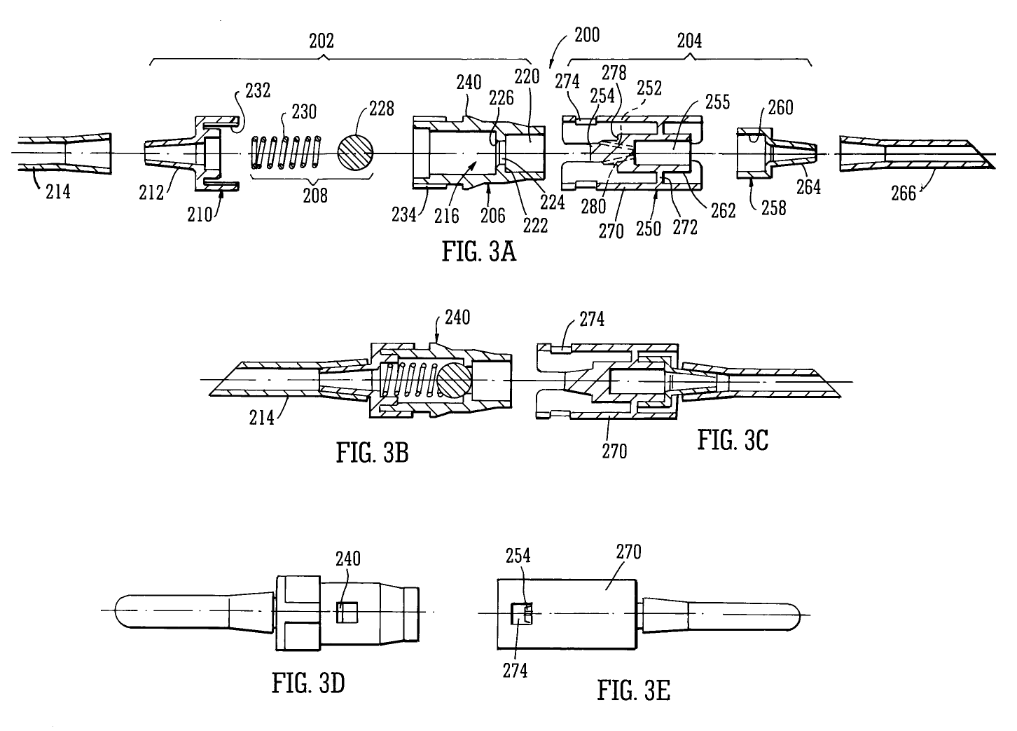

FIGS. 3A to 3E shows various views of a third embodiment of a connector according to the present invention; and

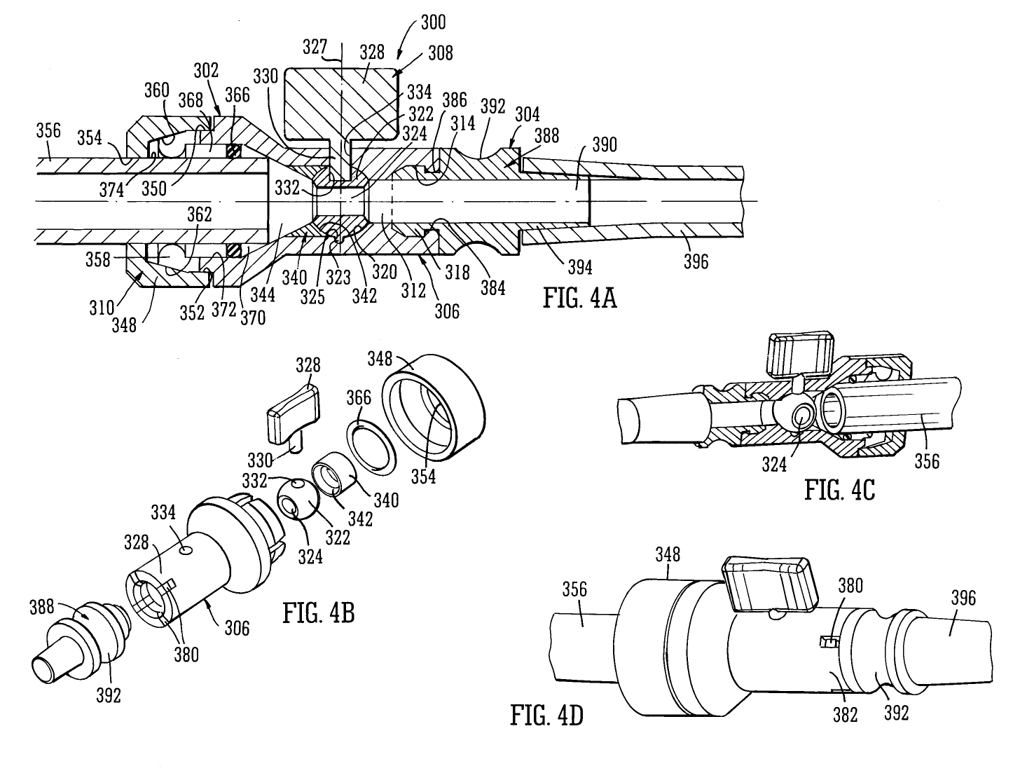

FIGS. 4A to 4D show various views of a fourth embodiment of a connector according to the present invention.

Referring now to the drawings and where the same features are denoted by common reference numerals.

FIGS. 1A to 1D show: an exploded sectional view of the components of a female connector portion at 1A; an exploded sectional view of a male connector portion at 1B; a view in elevation of a connector having the female and male connector portions connected at 1C; and, a perspective view of a connector of FIGS. 1A to 1C.

A connector is shown generally at 10 and comprises a male portion 12 and a female portion 14. The female connector portion 14 comprises a body portion 16 having a fluid flow passage 18 therethrough and a valve mechanism accommodating chamber 20 arranged obliquely to the flow passage 18. The flow passage 18 has an inlet/outlet 22 to which is attached an end cap 24 having a hollow conduit connecting spigot 28 for connection to a conduit 30 by push-fitting. The end cap 24 is an interference push-fit on a reduced diameter portion 32 of the body portion 16 an internal land 34 of the end cap 24 engaging with the reduced diameter portion 32 of the body portion. The female portion 14 has a self sealing valve 36 comprising a ball 38 a resilient biasing member shown as a spring 40 and an end cap 42 which engages with the body portion 16 at a reduced diameter portion 44 by means of co-operating threads 46, 48 The cap 42 and spring 40 keep the ball 38 engaged with a sealing seat 50 in the body portion 16 in the absence of an engaged male portion 12 and prevents any fluid flow through the conduit 30 and flow passage 18. The female portion 14 also has at an engaging end 52 an extending male collar portion 54 having a circular rebate 56 extending around the collar portion 54 at its junction with the main body portion 16 of the female portion 14. The male portion 12 comprises a male engaging portion 60 having a flow passage 61 therethrough, the portion 60 having a projecting tubular leading portion 62 and a co-operating female collar portion 64 to accept the male collar portion 54 of the female portion 14 in an axially directed cylindrical cavity 65 in the collar 64. The female collar portion 64 has a radially inwardly extending lip 66 which rides over the male collar portion 54 and engages the rebate 56 when the male portion 12 is fully engaged with the female portion 14. The male portion 12 has an end cap 68 which has a tubular connecting spigot 70 to accept a conduit 72 by push fitting in similar fashion to the end cap 24 of the female portion 14. A sealing “O” ring 74 is provided to seal the male and female portion 12, 14 together, the “O” ring being held and slightly compressed between radially directed faces 76, 78 on the female and male portions, respectively. On connection of the male and female connecting portions, the projecting tubular leading portion 62 which has an angled leading edge 80 enters the fluid flow passage 18 of the female portion and dislodges the ball 38 from its seat 50 and pushes it back up the cylindrical valve chamber 20 which has a larger diameter than the flow passage 18 which is too small to receive the ball 38. Dislodging the ball 38 from the seat 50 provides an uninterrupted fluid flow passage 18, 61 allowing fluid to flow between the conduits 30 and 72.

An advantage of this embodiment is that an unobstructed flow passage 18, 61 is produced through the engaged connector portions thus minimising the risk of blockages.

FIGS. 2A to 2D show: a cross section of engaged male and female connector portions having a resilient valve member therebetween at 2A; a perspective view of a female connector portion and it relationship to the resilient valve member at 2B; a perspective view in elevation of the connector of FIG. 2A at 2C; and, a perspective, partially sectioned view having “ghost” outlines of features of a resilient valve member at 2D.

A connector according to a second embodiment of the present invention is shown generally at 100 and comprises a female portion 102, a male portion 104 and a resilient valve member 106 sandwiched therebetween. The female portion 102 comprises a body portion 108 having a flow passage 110 therethrough. The body portion 108 has a frusto-conical recess 112 at a connection end with the male portion 104 and a hollow spigot portion 114 at a conduit 116 connecting end, the spigot having serrations 118 to aid conduit retention. The body portion has a radially outwardly directed upstanding lip 120 at an outward end of a reduced diameter portion 122 which encircles a part of the axial length of the frusto-conical recess 112, the reduced diameter portion 122 being terminated at a radially directed face 126. The male connector portion 104 has a body portion 130 has a fluid flow passage 132 therethrough and a male frusto-conical shaped projection 134 at it connection end with the female portion 102 and which male frusto-conical portion 134 co-operates with the female frusto-conical shaped recess 112 as explained hereinbelow. The male portion has a hollow spigot 136 for connection to a conduit 138 and which spigot 136 has serrations 140 to aid conduit retention. At a connection end with the female connector portion 102, the male body portion 130 has an axially extending collar 142 which has at its axial extremity a radially directed inturned lip 144 which, on coupling of the female 102 and male 104 connector portions, rides over the lip 120 and snaps into the reduced diameter portion 122 to retain the male and female connector portions together. The axially extending collar portion 142 terminates at a radially directed face 146 which, when the male an female portions are engaged abuts the radially directed face 126 of the female portion 102. The frusto-conical shaped recess 112 in the female portion 102 receives a valve member 106 which is formed from a resilient but elastic material able to accommodate large deformation but to regain its former shape when released. Suitable materials may include silicone rubbers, polyurethane, EVA and the like. The valve member 106 comprises a frusto-conical wall portion 150 of dimensions which suitably correspond to the internal dimensions of the recess 112 in the female body portion 108 and the external dimensions of the frusto-conical projection 134 of the male body portion 130. The valve member has an aperture 152 in the base thereof which generally in size corresponds to the flow passage 110 of the female connector portion, the aperture 152 being surrounded by a radially directed inturned flange portion 156 which is trapped, together with the wall 150, between the nose 158 of the frusto-conical projection 134 and the base of the frusto-conical recess 112 when the male and female portions are fully engaged. The valve member 106 is provided with a membrane 160 towards the wider end of the frusto-conical wall 150, the membrane having a slit 162 therein which when relaxed is closed and prevents fluid flow therethrough. When the male and female connector portion are joined together, the nose 158 of the male connector portion 104 pushes through the slit 162 in the valve member 106 to establish a fluid flow path through the flow passages 110, 132, the lip 144 being resiliently located in the reduced diameter portion 122 to hold the male and female connector portions in engagement. When the male and female connector portions are disconnected, and the male connector portion withdrawn from the valve member 106, the membrane 160 closes up about the slit 162 to prevent fluid flow from conduit 116 through the female connector portion 102. The valve member 106 may be adhesively bonded in the recess 112 so as to be retained therein when the connector is pulled apart. Alternatively, the valve member 106 may be over moulded into the recess 112 by means of a so-called 2-shot injection moulding process.

An advantage of this embodiment is that an unobstructed flow path 110, 132 is formed so minimising the risk of blockages.

FIGS. 3A to 3E show: an exploded cross sectional view of a connector according to a third embodiment of the present invention at 3A; a cross section of an assembled female connector portion of FIG. 3A at FIG. 3B; a cross section through an assembled male connector portion of FIG. 3A at FIG. 3C; a side elevation of a female connector portion of FIG. 3B rotated through 90⁹ at FIG. 3D; and, a side elevation of a male connector portion of FIG. 3C rotated through 90° at FIG. 3E.

A connector according to a third embodiment of the present invention is shown generally at 200 and comprises a female connector portion 202 and a male connector portion 204.

The female connector portion 202 comprises a body portion 206; a valve portion 208; and, an end cap portion 210 having a connecting hollow spigot 212 for connection to a conduit 214. The body portion 206 comprises a combined flow passage and valve chamber 216. An internal, radially inwardly directed flange member 222 having an aperture 224 therein forms a recess 220 for accepting a portion of the male connector portion as explained below. The recess 220 has a smaller diameter than the flow passage and valve chamber 216 which houses the valve portion 208. The edge of the aperture 224 facing the valve chamber 216 has a chamfer 226 which provides a sealing seat for a valve ball 228. The valve ball 228 is held in fluid flow sealing engagement (in the absence of an engaged male connector portion) with the seat chamfer 226 by a resilient biasing member, in this case a spring 230, the spring 230 being compressed by the end cap portion 210 when the latter is engaged with the body portion 206. Engagement of the cap portion 210 with the body portion 206 may be effected by any suitable means such as co-operating screw threads 232, 234 or a well known bayonet type of fitting having an engage and twist motion to lock, for example. The outer surface of the body portion 206 is provided with upstanding pips 240 which are received in co-operating recesses in corresponding features of the male connector portion 204 to hold the male and female connector portions together and to be explained below. The end cap portion 210 and the co-operating end of the body portion 206 have various features which serve to centralise the spring 230 and keep the cap 210 and body portion 206 concentric and which are apparent from FIGS. 3A and 3B but which will not be explained in detail. The male connector portion 204 comprises a body portion 250 which has a central projection 252 having a nose 254 of a diameter to pass through the aperture 224 in the female body portion 206 and a central fluid flow passage 255. The body portion has an end cap portion 258 which engages by interference between surfaces 260 and 262, the end cap portion having a hollow spigot 264 for connection to a conduit 266. The body portion also includes two arcuate shell-like clip members 270 which effectively resiliently pivot about pillars 272, the clip members 270 having apertures 274 therein which engage with the upstanding pips 240 on the female connector body portion 206. Engagement between the pips 240 and apertures 274 being effected by merely pushing the male and female connector portions together so that the clip members 270 resiliently rotate about the pillars 272 and snap closed when the pips 240 and apertures 274 are in register. When the male and female connector portions are fully engaged and the pips 240 and apertures 274 are engaged the projection 252 pushes the ball 228 away from its seat 226 to permit fluid flow through the flow passages 216, 255. When the male and female connector portions are fully engaged a shoulder 278 at the base of the projection 252 abuts the flange member 222. Fluid flow is permitted by the projection 252 having generally axially directed fluid flow apertures 280 therethrough which are exposed to the flow passage 216 when the connector portions are fully engaged and the ball valve 228 pushed off its seat 226 allowing fluid flow around the ball 228 and through the apertures 280 in the projection 252. Disconnection of the male and female connector portions is effected by pressing down on the ends of the clip members 270 (to the right of the pillars as shown in FIGS. 3A and 3C) to pivot the apertures 274 out of engagement with the pips 240 thus permitting the two connector portions to be separated and allowing the ball valve 228 to regain its sealing seat 226.

Instead of the generally axial flow apertures 280, the nose portion 254 may be provided with, for example, generally axially directed surface channels (not shown) in a cruciform array, for example.

FIGS. 4A to 4D show: a cross section through an assembled connector according to a fourth embodiment of the present invention at FIG. 4A; a perspective view of the components of the connector of FIG. 4A in exploded form at FIG. 4B; a part sectioned perspective view of the connector of FIG. 4A at FIG. 4C; and, a perspective view of the connector of FIG. 4A at FIG. 4D.

A fourth embodiment of a connector according to the present invention is shown generally at 300 and comprises a female connector portion 302 and a male connector portion 304. The female connector portion 302 comprises a body portion 306; a ball valve assembly 308; and, and end cap and conduit securing assembly 310. The body portion 306 has fluid flow passage 312 therethrough and a socket portion 314 for accepting an engaging nose piece 318 of the male connector portion 304. On the side of the flow passage 312 remote from the socket portion 314 there is a spherical seating portion 320 of the ball valve assembly 308, the seating portion 320 having seated therein a flow controlling ball valve 322 having an aperture 324 therethrough and which aperture can be turned to be in line with the flow passage 312 or through 90° so as to close off the flow passage 312 to fluid flow (the ball valve may be turned to any intermediate position between the two extremes so as effect a flow rate controlling effect) by a manually operated handle 328 which has a shaft 330 which engages a socket 332 in the ball valve 322, the shaft passing through the body portion 306 via an aperture 334. The ball valve 322 also has a pip 323 thereon which engages a dimple 325 in the body portion to ensure that the ball valve rotates about a constant axis 327. A separate ball valve seat insert 340 is also provided to support the side of the ball valve 322 opposite to the seat portion 320, the insert having a second seating portion 342 so as to fully support the ball valve 322 and a flow passage 344 therethrough. The end cap and conduit securing assembly comprises an end cap 348 which attaches to the body portion 306 by means of interference between two seating lands 350, 352 on the body portion 306 and end cap 348, respectively. The end cap 348 has an aperture 354 through which a conduit 356 passes and a split collett 358 which is radially compressed by mutually co-operating inclined surfaces 360, 362 on the end cap 348 and collett 358, respectively so as to grip the outer surface of the conduit 356. An “O” ring seal 366 is pushed by a cylindrical collar piece 368 abutting the collett 358 into a wedge shaped gap 370 so as to seal between an inner surface 372 of the body portion and the outer surface 374 of the conduit. The socket portion 314 of the female body portion 306 is provided with four axially directed slits 380 which renders the socket portion in the form of four axially directed fingers 382, each finger having and a radially inwardly directed lip portion 384 and which fingers 382 are able to resiliently deflect radially outwardly so as accept the nose piece 318 of the male connector portion 304, the lip portions 384 resiliently engaging a rebate 386 in a body portion 388 of the male connector portion 304. The body portion 388 has a fluid flow passage 390 therethrough which is in line with the flow passages in the female connector portion 306. The body portion 388 has a circular recess 392 to aid gripping by a user's fingers (not shown). A hollow spigot 394 is provided on the body portion to accept a soft plastics material conduit 396 pushed thereon.

An advantage of this embodiment is that an unobstructed flow path 344, 324, 312, 390 is formed through the assembled connector.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A connector for joining ends of two conduits, the connector comprising: a male connector portion and a female connector portion, both portions having conduit connection means for the connection of a conduit to each portion; engagement means to hold the male and female connector portions in fluid flow establishing connection when connected together and valve means associated with one of said connector portions.

2. A connector according to claim 1 wherein the male and female connector portions are held together by mutually engaging resilient means.

3. A connector according to claim 2 wherein the mutually engaging resilient means are snap fit means.

4. A connector according to claim 1 wherein at least one of the connector portions contains valve means.

5. A connector according to claim 4 wherein the female connector portion contains valve means.

6. A connector according to claim 4 wherein the valve means close automatically when the male and female portions are disconnected.

7. A connector according to claim 6 wherein the valve means comprises a resiliently biased ball valve.

8. A connector according to claim 7 wherein the ball valve is biased onto a seat to close a fluid flow passage in the female connector portion.

9. A connector according to claim 8 wherein the male portion has a projection which dislodges the ball valve from its seat to open a fluid flow passage through the connector.

10. A connector according to claim 9 wherein the projection pushes the ball valve back into a fluid flow passage allowing fluid flow around the ball valve.

11. A connector according to claim 9 wherein the projection has a fluid flow passage therein.

12. A connector according to claim 9 wherein the projection pushes the ball valve back into a non-fluid flow valve chamber.

13. A connector according to claim 5 wherein the valve means comprises a ball valve having a fluid flow aperture therethrough, the aperture being able to be turned to close or open a fluid flow passage in the connector.

14. A connector according to claim 13 wherein the ball valve has means for turning manually.

15. A connector according to claim 5 wherein the valve means comprises a resilient elastic diaphragm.

16. A connector according to claim 15 wherein the resilient elastic diaphragm comprises a self-closing slit in a membrane.

17. A connector according to claim 15 wherein the resilient elastic diaphragm is sandwiched between the male connector portion and the female connector portion.

18. A connector according to claim 15 wherein the elastic diaphragm is included in a frusto conical-shaped moulded valve member.

19. A connector according to claim 15 wherein the elastic diaphragm is opened by a nose portion of the male connector portion, the nose portion having a flow passage therethrough.

20. A connector according to claim 18 wherein the valve member is moulded with one of the male or female connector portions.

21. (canceled)
